(19) United States
(12) Patent Application Publication
(10) Pub. No.: US 2005/0251204 A1
(43) Pub. Date: Nov. 10, 2005

(54) Wound clamp

(76) Inventors: Jurg Attinger, Stein am Rhein (CH); Lorenz Egli, Neuhausen am Rheinfall (CH)

Correspondence Address: ALCON RESEARCH, LTD., R&D COUNSEL, Q-148, 6201 SOUTH FREEWAY, FORT WORTH, TX, 76134-2099, US

(21) Appl. No.: 10/840,123

(22) Filed: May 6, 2004

(51) Int. Cl. A61B 17/08

(52) U.S. Cl. 606/221

(57) ABSTRACT

A wound clamp having two substantially identical halves. The clamp halves are hinged and biased together using a spring. Prongs or needles integrally formed with the halves cause the clamp to be engaged firmly on the conjunctival and scleral tissue about the wound. The biasing of the clamp halves toward each other provide for a tight sealing of the wound.

### BACKGROUND OF THE INVENTION

This invention relates generally to the field of microsurgery and, more particularly, to ophthalmic microsurgery.

During ophthalmic microsurgery, it is often necessary to remove, dissect, cut, delaminate, coagulate or otherwise manipulate delicate tissues within the eye. Microsurgical tools, such as vitreous cutters, infusion cannulas, micro scissors, micro forceps, illuminated laser probes and other devices are generally used. These devices generally are inserted through one or more surgical incisions in the sclera, a sclerotomy, and different tools may be used during different parts of the surgical procedure. The repeated insertion and removal of these instruments can allow vitreous humor and fluids to escape the eye out through the sclerotomy, increasing the potential for softening of the globe, bleeding, traction on the retina and/or introduction of bacteria into the eye as well as increased healing time.

Therefore, a need continues to exist for a simple, inexpensive clamp for holding the sclerotomy tightly closed during surgery.

### BRIEF SUMMARY OF THE INVENTION

The present invention improves upon the prior art by providing a wound clamp having two substantially identical halves connected by a hinge. The clamp halves are hinged and biased using a spring. Prongs or needles integrally formed with the halves cause the clamp to be engaged firmly on the conjunctival and scleral tissue about the wound. The biasing of the clamp halves toward each other provide for a tight sealing of the wound.

Accordingly, one objective of the present invention is to provide a simple, inexpensive clamp for holding the sclerotomy tightly closed during surgery.

Another objective of the present invention is to provide a clamp have two clamp halves that are biased together using a spring or elastomeric band.

Another objective of the present invention is to maintain alignment of the conjunctiva and scleral incisions if trans-conjunctival surgery is performed.

Still another objective of the present invention is to facilitate introduction of the instruments through the surgical wound.

These and other advantages and objectives of the present invention will become apparent from the detailed description and claims that follow.

### BRIEF DESCRIPTION OF THE DRAWING

FIG. 1 is a top, left and rear perspective view of the wound clamp of the present invention with the prongs removed.

FIG. 2 is a bottom, left and front perspective view of the wound clamp of the present invention with the prongs removed.

FIG. 3 is a cross-sectional view of the wound clamp of the present invention shown in the open position.

FIG. 4 is a cross-sectional view of the wound clamp of the present invention shown in the closed position.

### DETAILED DESCRIPTION OF THE INVENTION

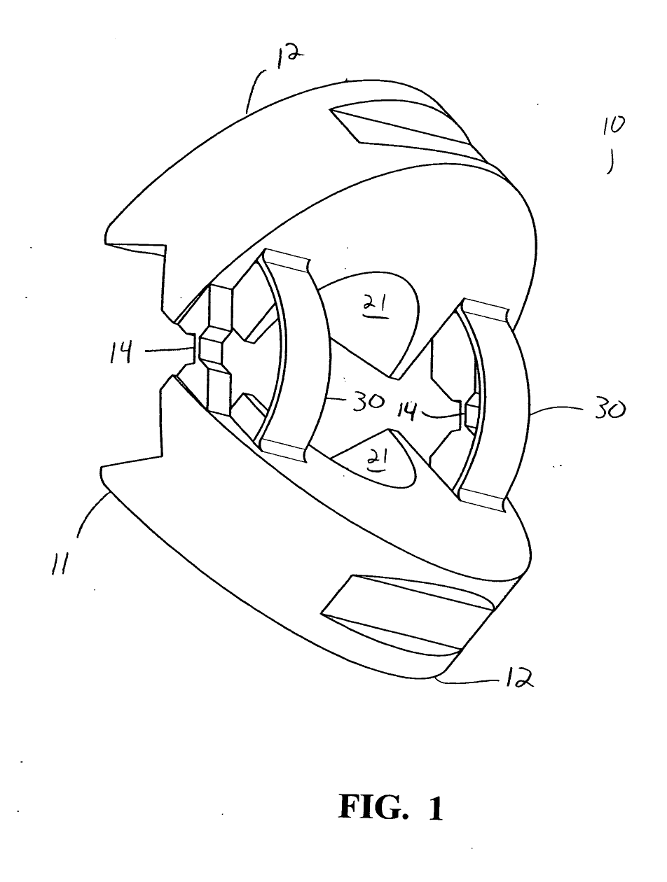

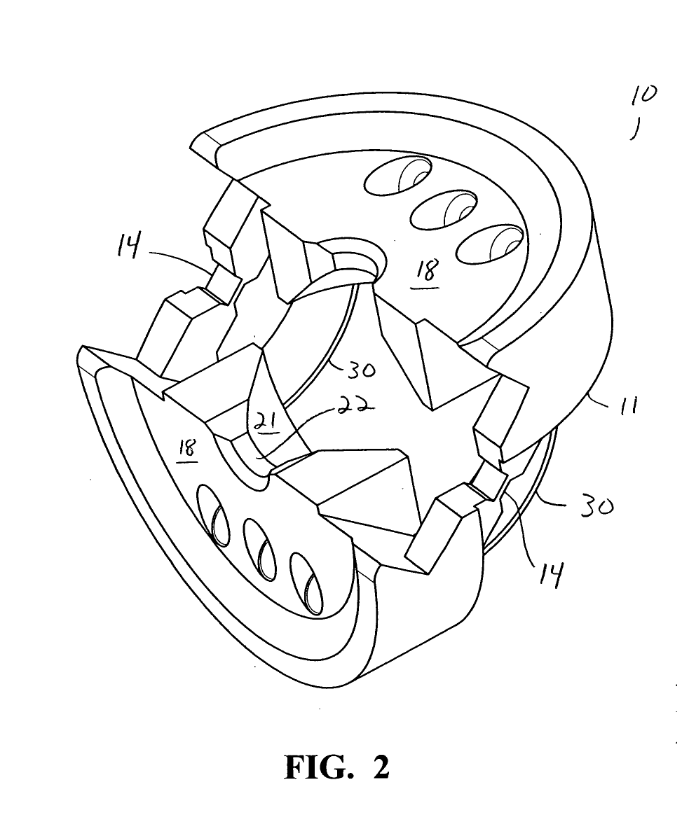

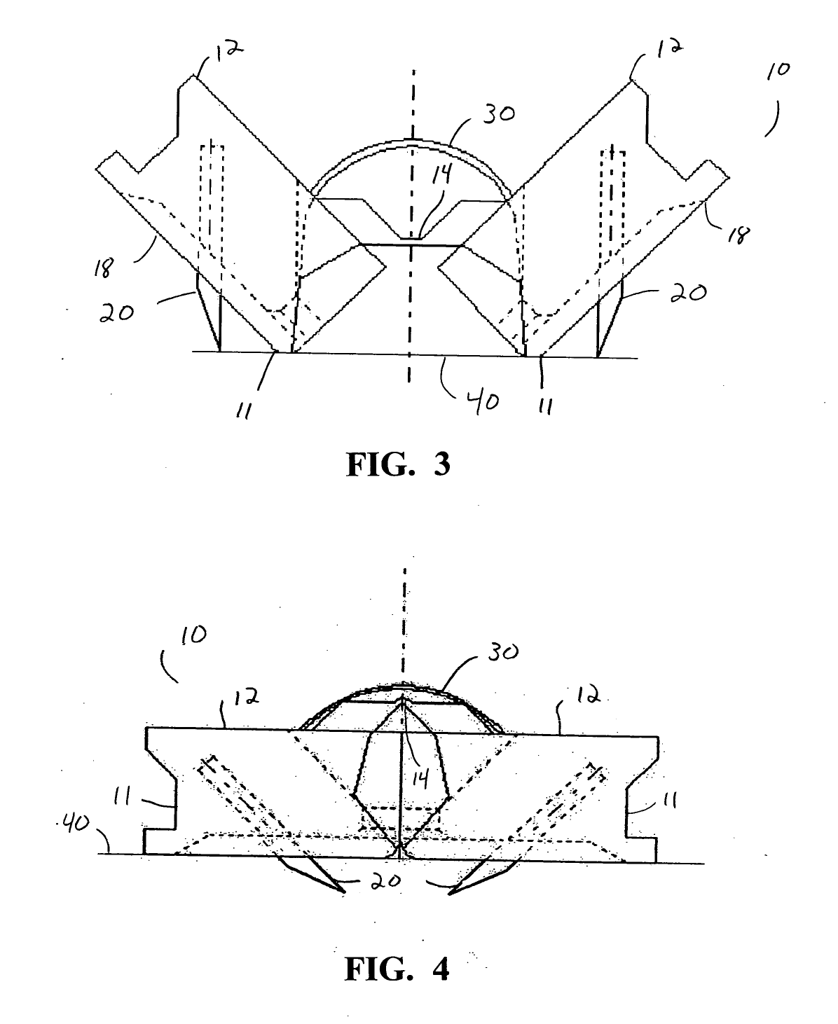

As best seen in FIGS. 1 through 4, wound clamp 10 generally consists of body 11 made up of symmetrical halves 12 connected by hinges 14 and snap springs 30. Preferable, halves 12, hinges 14 and springs 30 are integrally formed as one piece. As best seen in FIGS. 3 and 4, bottom 18 of halves 12 may contain a plurality of hooks or prongs 20. Clamp 10 may be molded or formed from any suitable material, such as thermoplastic, and may contain a lubricious material, such as polytetrafluoroethylene (TEFLON®), and/or a luminous material so as to aid in visualizing clamp 10 in a darkened room. Each of halves 12 contain cut out portions 21 that together define central aperture 22 when halves 12 are biased together. Aperture 22 allows a surgical tool (not shown) to be inserted into a wound when clamp 10 is placed over a surgical site and halves 12 pulled together by springs 30. Portions 21 may also be flared, beveled or chamfered to assist in the introduction of the surgical tool into the wound. Prongs 20 may be made from any suitable material, such as stainless steel, suture materials such as polypropylene or modified surgical staples and may be integrally formed within halves 12 during the molding process or attached to halves 12 by any suitable process, such as adhesive.

In use, halves 12 of clamp 10 are forced apart about hinges 14 so that springs 30 hold clamp 10 in the open position shown in FIG. 33. Clamp 10 is placed over a surgical incision so that prongs 20 contact tissue 40 around the incision site. Halves 12 are then rotated about hinges 14 until springs 30 snap or pull halves 12 together as shown in FIG. 4, thereby forcing prongs 20 in tissue 40 around the incision site, causing clamp 10 to engage firmly on tissue 40 about the incision site and holding the incision closed. Surgical tools may be inserted into the incision through aperture 22 without causing excessive opening of the incision. In addition, halves 12 may spread apart slightly to enlarge aperture 22 without prongs 20 becoming dislodged from the tissue so as to allow foreign bodies or slightly larger instruments to exit the wound. To remove clamp 10, halves 12 are pivoted about hinges 14 until springs 30 pull or snap clamp 10 into the open position as shown in FIG. 3, thereby releasing prongs 20 from tissue 40.

This description is given for purposes of illustration and explanation. It will be apparent to those skilled in the relevant art that changes and modifications may be made to the invention described above without departing from its scope or spirit.

1. A wound clamp, comprising:
a) a body having a first half and a second half;
b) at least one hinge connecting the first half to the second half;
C) at least one spring biasing the first half against the second half about the hinge; and
d) a plurality of prongs associated with both the first half and the second half.

2. The wound clamp of claim 1 wherein first half and the second half each contain cut out portions that form an aperture when the first half and the second half are biased together by the spring.

3. The wound clamp of claim 1 wherein the body comprises a lubricious material.

4. The wound clamp of claim 1 wherein the body comprises a luminous material.

5. The wound clamp of claim 2 wherein the cut out portions are flared.

6. The wound clamp of claim 2 wherein the cut out portions are beveled.

7. The wound clamp of claim 2 wherein the cut out portions are chamfered.
